(19) United States
(12) Patent Application Publication
(10) Pub. No.: US 2006/0127960 A1
(43) Pub. Date: Jun. 15, 2006

(54) Process for preparation of an agglutination reagent for rapid detection of typhoid

(75) Inventors: Ganga Prasad Rai, Gwalior (IN); Gauri Shankar Agarwal, Gwalior (IN); Shri Krishna Sharma, Gwalior (IN); Devendra Kumar Jaiswal, Gwalior (IN); Krishanamurthy Shekhar, Gwalior (IN); Kajal Arora, New Delhi (IN); Vijay Kumar Chaudhary, New Delhi (IN)

Correspondence Address: THE WEBB LAW FIRM, P.C., 700 KOPPERS BUILDING, 436 SEVENTH AVENUE, PITTSBURGH, PA, 15219, US

(73) Assignee: DEFENSE RESEARCH & DEVELOPMENT ORGANISATION, NEW DELHI (IN)

(21) Appl. No.: 10/536,533

(22) PCT Filed: Sep. 3, 2003
(86) PCT No.: PCT/IN03/00296

§ 371 Date: Nov. 29, 2005

(30) Foreign Application Priority Data
Nov. 26, 2002 (IN) 1187/DEL/02
Mar. 12, 2003 (IN) 271/DEL/03

(51) Int. Cl. G01N 33/554; G01N 33/569

(52) U.S. Cl. 435/7.32

(57) ABSTRACT

A process for the preparation of an agglutination reagent for rapid and early detection of typhoid, comprising the steps of: preparing Salmonella typhi specific antibody; preparing latex particles suspension; and coating said latex particles with the antibody.

### FIELD OF INVENTION

The present invention relates to a process for the preparation of an agglutination reagent for rapid and early detection of Salmonella typhi in serum.

### PRIOR ART

Typhoid is an endemic febrile disease caused by Salmonella typhi. Typhoid is a major concern of public health. The organism usually enters the body by consumption of contaminated food or water and penetrates the intestinal wall. After that if multiplies and enters blood stream within 24-72 hours resulting in enteric fever and bacteremia. After an incubation period of 10 to 14 days, early symptoms of typhoid, like headache, fever, loss of appetite, bradycardia, splenomegaly etc. appear. Typhoid is diagnosed either by blood culture or by detection of its antigens or by the detection of its antibodies in the blood.

One of the most adapted methods for diagnosing the typhoid fever is the performance of “Widal test”, a serological test based on the detection of antibodies in the blood. This test is based on the fact that antibodies against typhoid, remain in the blood of infected person, bind to the bacteria and results in the clumps formation which is referred as “Widal Agglutination”.

One of the imitation of the widal test is that the test is not specific as i cross reacts with other febrile organisms and many organisms of family Enterobacteriaceae.

Another limitation of the widal test is that, as typhoid is an endemic disease hence there always exist some background level of antibody in the endemic areas. Hence it becomes necessary to determine the cut-off titra for each region to rule out the possibility of diagnosis as false positive.

Yet another limitation of the widal test is that it gives position results only after one or two weeks of the onset of fever.

Still another limitation of the widal test is that test it is to be performed on paired serum samples taken at an interval of at least one week apart because single widal test is elusive and inconclusive.

Further limitation of the widal test is that the antibiotic administration in the early phase of infection, inhibits the development of the antibody and hence test may give false negative result.

Still further limitation of the widal test is that TAB vaccinated normal healthy persons give false positive reaction in widal test due to presence of circulating antibody against vaccine in human system.

Another limitation of the widal test is that it gives indirect evidence of typhoid infection.

Further limitation of the widal test is that the test has low sensitivity and low specificity.

Other technique known for diagnosis of typhoid is based upon isolation and identification of causative agent. This procedure is termed as golden standard.

In this technique Salmonella typhi is isolated from blood and identified by microscopic and biochemical tests. However, this technique has many limitations.

One limitation of the above technique is that it is time consuming as it requires long period of incubation from 3 days to 14 days and also requires elaborate laboratory facilities.

Another limitation of the above technique is that for its performance large quantity of blood sample (10 ml/patient) is required.

Yet another limitation of the above technique is that it needs large volume of culture medium i.e. 100 ml (10 times of blood sample).

Still another imitation of the above technique is its low sensitivity (40 to 60%), as there are very few organism in circulation, as low as 1/ml which leads to false negative results.

Further limitation of above method is that bacterial growth in culture is inhibited by serum bactericidal agents, present in blood which may lead to false negative results.

Still further limitation of blood culture is that antibiotics treatment during early phase of infection may inhibit bacterial growth in culture which may give false negative results.

Other known techniques such as Radioimmunoassay (RIA), Enzyme—linked immunosorbent assay etc. are based on detection of circulating antigen in the body fluids, but these techniques have many limitations.

One limitation of these techniques is that they require sophisticated and elaborate laboratory facilities.

Another limitation of RIA is that K requires radioactive material which is health hazard and also needs trained personnel to handle the radioactive material.

Still further limitation of above techniques is that reagents are expensive.

Further limitation of these techniques is that minimum 4-5 hours are required to perform the tests.

### OBJECT OF THE INVENTION

The primary object of the present invention is to provide a process for the preparation of an agglutination reagent for rapid and early detection of S.typhi in serum samples of suspected typhoid patients.

Another object of the present invention is to provide a process for the preparation of an agglutination reagent wherein the proposed reagent enables diagnosis of typhoid within 3 minutes after collection of serum samples.

Yet another object of the present invention is to provide a process for the preparation of an agglutination reagent wherein the serum sample required for the diagnosis of typhoid disease is as small as 20 μl.

Further object of the present invention is to provide a process for the preparation of an agglutination reagent wherein the reagent enables the detection of typhoid bacteria by simple latex agglutination technique.

Still further object of the present invention is to provide a process for the preparation of an agglutination reagent wherein the reagent enables specific identification of Salmonella typhi antigen in serum samples of suspected typhoid patients.

Yet further object of the present invention is to provide a process for the preparation of an agglutination reagent wherein the reagent enables the diagnosis of typhoid in the early stages of infection even within one or two days after the on set of the fever.

Another object of the present invention is to provide a process for the preparation of an agglutination reagent wherein the reagent is highly sensitive.

Yet another object of the present mention is to provide a process for the preparation of an agglutination reagent which enables diagnosis of the disease in field conditions as it does not require any equipment or laboratory facility.

Still further object of the present invention is to provide a process for the preparation of an agglutination reagent that does not require any specially trained personnel to perform the test.

Yet further object of the present invention is to provide a process for the preparation of an agglutination reagent which enables the diagnosis of those patients who have been administered with antibiotics resulting in blood culture isolation as negative.

### DESCRIPTION OF PROCESS

According to the preferred embodiment of the present invention, the agglutination reagent is prepared by a process comprising of following steps:

(a) Preparation of Antibody (Immunoglobulins):

Flagellin geme sequence specific to Salmonella typhi is cloned and expressed by recombinant DNA technology. The expressed recombinant protein is purified by affinity chromatography. Hyper immune sera against this recombinant protein is raised in rabbit. Immunoglobulin fraction of hyper immune sera is separated by ammonium sulphate precipitation. The precipitated immunoglobulins are suspended in 50 mM phosphate buffer (pH 7.2), dialysed and protein content determined.

(b) Preparation of Latex Particles Suspension:

1% carboxylated latex particles of size 0.88 to 0.90 μm and 40 mM 2-N Morphilinoethane sulphonic acid (MES) buffer (pH 5.5-6.0) are taken in a preferred ratio of 1:1 in a tube. They are mixed on a vortex mixer for around 60 seconds and centrifuged at 10,000 rpm for 10-12 minutes at about 4° C. The latex particles are further washed twice in 20 mM MES buffer of pH 5.5 by mixing on vortex mixer for around 60 seconds, followed by centrifugation at 10,000 rmp for 10-12 minutes at about 4° C. After the final wash, the latex particles are suspended in 20 mM MES buffer of pH 5.5 and the volume is made up equal to the starting volume of the latex particles. The suspension is then sonicated by a tip sonicator at about 5 watts for 60-120 seconds, preferably 90 seconds. To this suspension freshly prepared solution of 0.1 M1-ethyl-3(3-dimethyl-amino-propyl)carbodimide hydrochloride (EDC) in 20 mM MES buffer (pH 5.5) taken in the preferred ratio of 1:1, is added drop wise, while the solution is slowly vortexed. The tube is rotated slowly end-over-end for about 3 hours at a temperature of 20-25° C. It is then washed thrice with 20 mM MES buffer (pH 5.5) at 10,000 rpm for 10-12 minutes at a temperature of about 4° C. The latex particles are resuspended in MES buffer (20 mM, pH 5.5) and sonicated for 60-120 seconds by a tip sonicator at 5 watts.

(c) Coating of Latex Particles with Antibody (Immunoglobulins):

To the suspension of latex particles prepared in step (b), 0.6-1.0 mg preferably 0.8 mg per ml of the suspension, immunolobulins prepared in step (a) are added. The whole mixture is then rotated end-over-end for 18-20 hours at a temperature of 20-25° C. The coating reaction is stopped by addition of 1M glycine (pH 11.0) taken in quantity of 0.06 ml per ml of solution of immunoglobulin coated latex particles. Rotation is continued for about 30 minutes at a temperature of 20-25° C. The coated latex particles are pelleted out by centrifugation at 10,000 rpm for 10-12 minutes at a temperature of about 4° C. The pellet is washed thrice with washing buffer (50 mM glycine, pH 8.5; 0.03% triton X-100 and 0.05% sodium azide) at 10,000 rpm for 10-12 minutes at a temperature of about 4° C. Finally the washed coated latex particles are resuspended in storage buffer (50 mM glycine, pH 8.5; 1.0% bovine serum albumin; 0.03% triton X-100; 0.1% sodium azide and 0.01% thiomersol to a final concentration of 1% and sonicated by a tip sonicator for around 60 seconds at about 5 watts and stored at 4° C.

### METHOD OF USE

(a) Take 20-40 μl (1 to 2 drops) of test serum, positive and negative controls at three distinct places on a glass slide

(b) Add 10-2 μl (1-2 small drops) of latex reagent to test serum, positive and negative controls

(c) Mix the reactants with separate wooden sticks carefully to avoid any intermixing of reactant placed at separate places and rotate the slide for 1-2 minutes.

A positive reaction is indicated by the development of an agglutination within 1-2 minutes of mixing the reagent with the test sample and positive control, showing clearly visible clumping of the particles. The speed of appearance and quality of agglutination depends on the strength of the antigen present, varying from large clumps which appear within a few seconds of mixing, to small clumps which develop rather slowly. In negative reaction the reagent does not agglutinate and the cloudiness or the turbid nature remains substantially unchanged throughout the lest.

Laboratory studies on the reliability of proposed agglutination reagent for rapid detection of Salmonella typhi in typhoid patient serum is performed with the laboratory strains of Salmonella typhi; and culture proven and widal positive serum samples collected from suspected cases of typhoid; and with serum samples of apparently normal healthy individuals. The result indicate 93.00% senshtWty and 98.00% specificity.

The present invention will now be illustrated with a working example which is intended to be illustrative example and is not intended to be taken restrictively to imply any limitation on the scope of the present invention.

### WORKING EXAMPLE

Flagellin gene sequence specific to salmonella typhi was amplified by polymerase chain reaction (PCR) using gene specific primers. Amplified PCR product was cloned in Glutathione-S-transferase (GST) vector and later expressed. The expressed protein was purified by GST affinity column chromatography. The protein content of the purified product was determined by Bradford method. Hyper immune serum against this protein was raised in rabbit. Immunoglobulins fraction of hyper immune sera was separated by ammonium sulphate precipitation. The precipitated immunoglobulins were suspended in 1.0 ml PB (50 mM, pH 7.2), dialysed and protein content determined. 1.0 ml of 1% carboxylated latex particles and 1.0 ml of 40 mM MES buffer (pH 5.5-6.5) were taken in 2.0 ml microcentrifuge tube. Then they were mixed on vortex mixer for 60 seconds and centrifuged at 10,000 RPM for 10-12 minutes at a temperature of 4° C. The latex particles were further washed twice in 2.0 ml of 20 mM MES buffer (pH 5.5) by mixing on vortex mixer for 60 seconds and centrifugation at 10,000 RPM for 10-12 minutes at a temperature of 4° C. Following the final wash, the latex particles were suspended in 1.0 ml MES buffer (20 mM, pH 5.5) and sonicated by a tip sonicator at 5 watts for 60-120 seconds. Later 1.0 ml of freshly prepared solution of 0.1 M 1-ethyl-3-(3-dimethylaminopropyl)carbodimide hydrochloride (EDC) in MES buffer (20 mM, pH 5.5) was added drop wise while the solution was slowly vortexed. Then the tube was rotated slowly end-over-end for 3 hours at a temperature of 20-25° C. followed by washing three times with MES buffer (20 mM, pH 5.5) at 10,000 RPM for 10-12 minutes at a temperature of 4° C. The latex particles were resuspended in 0.7 ml MES buffer (20 mM, pH 5.5) and sonicated for 60-120 seconds by a tip sonicator at 5 watts. 0.8 mg of immunoglobulins were added to latex particles and volume was made up to 1.0 ml with MES buffer (20 mM, pH 5.5). This was then rotated end-over-end for 18-20 hours at a temperature of 20-25° C. The coating reaction was then stopped by addition of 0.06 ml of 1M glycine (pH 11.0). The rotation was continued for 30 minutes at a temperature of 20-25° C. The coated latex particles were pelleted out by centrifugation at 10,000 RPM for 10-12 minutes at a temperature of 4° C. The pellet was washed thrice with 2.0 ml of washing buffer (50 mM glycine, pH 8.5, 0.03% triton X-100 and 0.05% sodium azide) at 10,000 RPM for 10-12 minutes at a temperature of 4° C. The washed coated latex particles were resuspended in storage buffer (50 mM glycine, pH 8.6. 1.0% bovine serum albumin, 0.03% triton X-100, 0.1% sodium azide and 0.01% thiomersol) to a final concentration of 1% and sonicated with tip sonicator for 60 seconds at 5 watts and stored at 4° C.

It is to be understood that the present invention is susceptible to modifications, changes and adaptations by those skilled in the art. Such modifications, changes, adaptations are intended to be within the scope of the present invention which is further set forth under the following claims:

1-22. (canceled)

23. A process for the preparation of an agglutination reagent for rapid and early detection of typhoid comprising:
(a) preparing antibody specific to Salmonella typhi;
(b) preparing latex particles suspension;
(c) coating of the said latex particles with the said antibody;
wherein the said process of preparing antibody specific to Salmonella typhi comprises cloning Flagellin gene sequence specific to Salmonella typhi, expressing the said Flagellin gene sequence by recombinant DNA technology, followed by purifying recombinant protein by affinity chromatography, raising the hyper immune sera against purified recombinant protein in animals like rabbit, separating the antibody (immunoglobulin) fraction of hyper immune sera by precipitating in ammonium sulphate, suspending in 50 mM phosphate buffer of pH 7.2 and dialyzing;
wherein the said process of preparing latex particle suspension comprises:
(i) mixing 1% carboxylated latex particles of size 0.88 to 0.90 μm and 40 mM 2-N Morphilinoethane sulphonic acid (MES) buffer of pH 5.5 to 6.0 in a ratio of 1:1 on a vortex mixer for about 60 seconds, centrifuging at 10,000 rpm for 10-12 minutes at about 4° C., followed by washing twice with 20 mM MES buffer of pH 5.5 at 10,000 rpm for 10-12 minutes at about 4° C., sonicating by a tip sonicator at about 5 watts for 60-120 seconds;
(ii) adding drop wise a freshly prepared solution of 0.1 M 1-ethyl-3 (3-dimethyl-amino propyl)carbodimide hydrochloride (EDC) in 20 mM MES buffer of pH 5.5 to the said sonicated latex particles obtained from step (i) above in a ratio of 1:1 while vortexing the suspension slowly, rotating the suspension slowly end-over-end for about 3 hours at a temperature of 20-25° C., washing thrice with 20 mM MES buffer (pH 5.5) followed by sonicating the washed suspension of latex particles by a tip sonicator for 60-120 seconds at about 5 watts;
wherein the said process of coating of the said latex particles is done by adding 0.6-1.0 mg preferably 0.8 mg per ml of the said antibody (immunoglobulins) to the said latex particle suspension, rotating the suspension end-over-end for 18-20 hours at a temperature of about 20-25° C., stopping the coating reaction by 1M glycine (pH 11.0) taken in quantity of 0.06 ml per ml of solution of antibody coated latex particles followed by centrifugation at 10,000 rpm for 10-12 minutes at a temperature of about 4° C., washing thrice with washing buffer comprised of 50 mM glycine, pH 8.5; 0.03% triton X-100 and 0.05% sodium azide, suspending in storage buffer to a final concentration of 1%, sonicating for about 60 seconds at about 5 watts and storing at 4° C.

24. An agglutination reagent for rapid and early detection of typhoid, comprising of 1% carboxylated latex particles coated with antibody specific to Salmonella typhi, suspended in storage buffer.

25. The agglutination reagent as claimed in claim 24, wherein the size of the said latex particles is 0.88 to 0.90 μm.

26. The agglutination reagent as claimed in claim 24, wherein the said storage buffer is comprised of 50 mM glycine pH 8.5, 1.0% bovine serum albumin, 0.03% triton X-100, 0.1% sodium azide and 0.01% thiomersal.

27. The agglutination reagent for rapid and early detection of typhoid as claimed in claim 24, wherein the said antibody is the immunoglobulin fraction, of the hyper immune sera raised in rabbit against the recombinant protein expressed by cloning of Flagellin gene sequence specific to Salmonella typhi by recombinant DNA technology, suspended in 50 mM phosphate buffer.

28. A kit for rapid and early detection of typhoid comprising 1% agglutination reagent as claimed in claim 24 suspended in storage buffer, glass slides, droppers, wooden sticks and positive and negative controls.
